(19) United States
(12) Patent Application Publication
(10) Pub. No.: US 2010/0256669 A1
(43) Pub. Date: Oct. 7, 2010

(54) Helical Vena Cava Filter

(75) Inventors: Keith S. Harris, Chandler, AZ (US); Robert G. Lerdahl, Phoenix, AZ (US); Khoi Q. Ta, San Jose, CA (US)

Correspondence Address: C. R. Bard, Inc.;Bard Peripheral Vascular, Inc., 1415 W. 3rd Street, P.O. Box 1740, Tempe, AZ, 85280-1740, US

(73) Assignee: C.R. Bard, Inc., Murray Hill, NJ (US)

(21) Appl. No.: 12/095,700

(22) PCT Filed: Nov. 28, 2006
(86) PCT No.: PCT/US06/45738

§ 371 Date: Jun. 17, 2010

Related U.S. Application Data
(60) Provisional application No. 60/742,148, filed on Dec. 2, 2005.

(51) Int. Cl. A61F 2/01

(52) U.S. Cl. 606/200

(57) ABSTRACT

A vena cava filter is described, having at least one member arranged helically along a longitudinal axis of the filter. The filter may include a plurality of legs around which is arranged one or more filaments traveling in a helical path, or an elongated wire member arranged to define a first and second helecal path. The filter may include hooks and/or a retreival member.

### PRIORITY

This application claims the benefit of priority to U.S. Application No. 60/742,148, filed Dec. 2, 2005, which is incorporated by reference into this application as if fully set forth herein.

### BACKGROUND

Inferior vena cava (IVC) filters are devices configured for insertion into the inferior vena cava to capture particles that may be present in the blood stream which, if transported to, for example, the lungs could result in serious complications and even death. Typically, IVC filters are utilized in patients who have a contraindication to anticoagulation or in patients developing clinically apparent deep vein thrombosis (DVT) and/or pulmonary embolism (PE). Patients who have recently suffered from trauma, have experienced a heart attack (myocardial infarction), or who have undergone major surgical procedure (e.g., surgical repair of a fractured hip, etc.) may develop clinically apparent DVT. When a thrombus clot loosens from the site of formation and travels to the lung, it may cause PE, a life-threatening condition. An IVC filter may be placed in the circulatory system to intercept one or more clots and prevent them from entering the lungs. IVC filters are either permanent or retrievable.

There are many different configurations for IVC filters, including those that include a central hub from which extend a plurality of struts that form filter baskets having a conical configuration, such as disclosed in U.S. Pat. No. 6,258,026, which is incorporated by reference in its entirety into this application. Other IVC filter configurations utilize wires and/or frame members to form straining devices that permit flow of blood while trapping larger particles. IVC filters are generally configured for compression into a small size to facilitate delivery into the inferior vena cava and subsequent expansion into contact with the inner wall thereof. The IVC filter may later be retrieved from the deployed site by compressing the legs, frame members, etc., depending on the filter configuration. Typically, an IVC filter will include hooks or anchoring members for anchoring the filter in position within the inferior vena cava. The hooks may be more elastic than the legs or frame members to permit the hooks to straighten in response to withdrawal forces, which facilitate withdrawal from the endothelium layer of the blood vessel without risk of significant injury to the vessel wall.

The following references relate to IVC filters: U.S. Pat. No. 5,059,205; U.S. Pat. No. 5,893,869; U.S. Pat. No. 6,059,825; U.S. Pat. No. 6,497,709; U.S. Pat. No. 6,517,559; U.S. Pat. No. 6,623,506; US Publication No. 2002/0193828; US Publication No. 2004/0073252; US Publication No. 2004/0158273; US Publication No. 2004/0230220; US Publication No. 2005/0055045; and US Publication No. 2005/0131451, which are incorporated by reference in their entireties into this application.

Applicants have recognized that it would be desirable to provide an IVC filter that includes one or more members arranged helically along a longitudinal axis of the IVC filter, and embodiments of such an IVC filter are described herein.

### BRIEF SUMMARY OF THE INVENTION

Accordingly, blood vessel filters are described herein, having one or more members arranged helically along a longitudinal axis of the filter. In one embodiment, a blood vessel filter includes a plurality of legs extending radially outward from a proximal portion of the filter along a longitudinal axis, the legs including a hook on a distal end thereof, and a filament connecting the legs, the filament traveling in a first helical path around the legs along the longitudinal axis.

In another embodiment, a blood vessel filter includes an elongated wire member arranged helically along a longitudinal axis, wherein both a proximal free end of the elongated wire member and a distal free end of the elongated wire member are positioned at a proximal end of the filter, the elongated wire member radially expanding along a first helical path from the proximal free end of the elongated wire member to a distal end of the filter and radially decreasing along a second helical path from the distal end of the filter to the distal free end of the elongated wire member.

These and other embodiments, features and advantages will become more apparent to those skilled in the art when taken with reference to the following more detailed description of the invention in conjunction with the accompanying drawings that are first briefly described.

### BRIEF DESCRIPTION OF THE DRAWINGS

FIG. 1 is a side perspective view of one embodiment of a blood vessel filter, including a filament helically arranged around filter legs.

FIGS. 2A-2F illustrate an alternative embodiment of FIG. 1 and a retrieval or repositioning procedure.

FIGS. 3A and 3B are a side perspective views of another embodiment of a blood vessel filter, including an elongated wire member defining a first and second helical path.

FIGS. 4A and 4B are photographs of an animal study for a filter utilizing hooks similar to those shown and described in the embodiments of FIGS. 1-2.

### DETAILED DESCRIPTION OF THE INVENTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

The filter embodiments discussed below may be used for insertion into the inferior vena cava or other blood vessels or cavities in a mammalian body. As used herein, the term “suture material” means a material that is, or could be, used as a suture thread by a surgeon, including, for example, synthetic polymers, polyglycolic acid (PGA), polydioxanone (PDS), polyglactin, nylon, polypropylene (prolene), silk, catgut, non-absorbable/non-biodegradable materials, and combinations thereof. Included in the term “suture material” are both monofilament and multifilament arrangements. Also, as used herein, the term “hook” denotes any suitable mechanism to connect the filter to the biological tissue such as, for example, a hook, a rod with barbs, double hooks, or arrow heads. Examples of hooks are provided in U.S. Pat. No. 6,258,026, which is incorporated by reference in its entirety into this application.

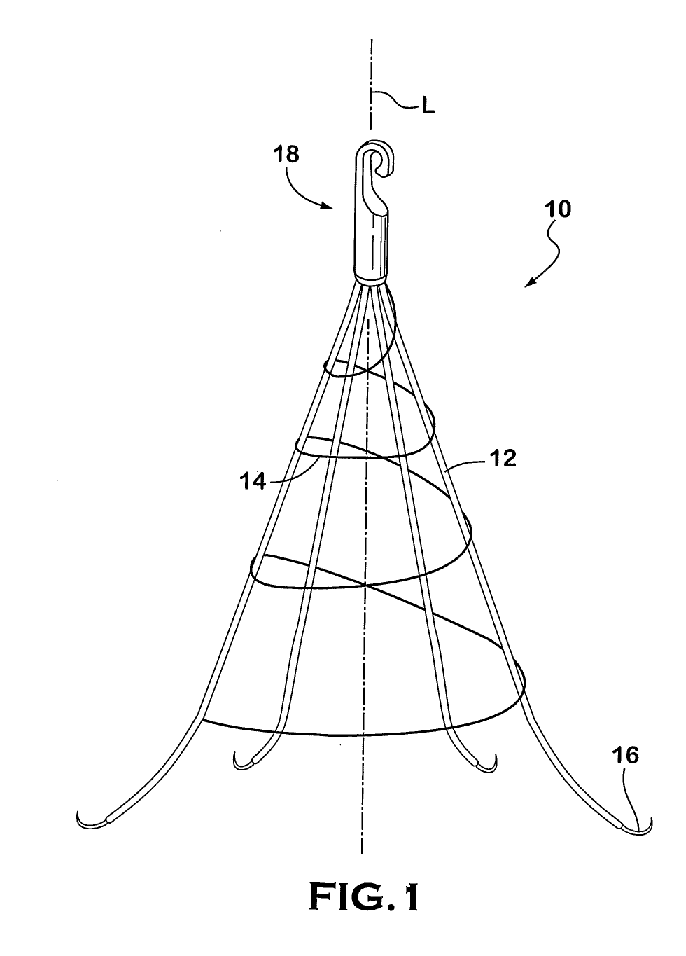

Referring to FIG. 1, an embodiment of a filter is shown. Filter 10 includes a plurality of legs 12 extending from a hub 18 at the proximal end of the filter 10, the legs 12 being attached together and also to the hub 18, or individually attached to the hub 18. The hub 18 is shown having a configuration of a retrieval member with a hook-like design, although in other embodiments, the hub forms a sleeve as known to one skilled in the art. The legs 12 extend radially outward from the hub 18 along a longitudinal axis L of the filter 10 in an expanded configuration to form a conical basket. The legs 12 may be individual wire members made of a material, such as, for example, stainless steel, shape memory metals, shape memory alloys, super elastic shape memory metal alloys, metal alloys, linear elastic shape memory alloy, shape memory polymers, polymers, and combinations thereof. The legs 12 may also be made of a bio-resorbable material such as, for example, the materials shown and described in U.S. Pat. No. 6,287,332 and U.S. Patent Application Publication No. 2002/0004060, which are incorporated by reference in their entireties into this application. The number of legs 12 of filter 10 can be wide-ranging (e.g., 2, 3, 4, 6, 12, etc.), but in a preferred embodiment, the filter 10 contains six legs.

The legs 12 may be circumferentially spaced equidistant from one another or may be otherwise arranged in an unbalanced configuration. In one embodiment, the legs 12 have a length that is approximately equivalent to one another, but in other embodiments, the legs have different lengths. For example, a first set of legs 12 could have a first length and a second set of legs 12 could have a second length greater than the first length. In this example, each of the first set of legs could be positioned between successive second set of legs so that the lengths of the legs alternate between a first length and second length about the circumference of the filter. Of course, numerous alternate configurations are possible with respect to the lengths and arrangements of legs 12, as one skilled in the art would appreciate, and such alternate configurations are within the scope of the invention. The legs 12 of filter 10 are shown in an expanded configuration, defining an expanded perimeter of the filter 10. For delivery of the filter 10 to a blood vessel, the legs 12 are compressed to a collapsed configuration, defining a collapsed perimeter of the filter 10 smaller than the expanded perimeter of the filter 10.

Attached to the distal end of each of the legs 12 is a hook 16 in the embodiment shown in FIG. 1, however in other embodiments, a hook 16 may be attached to fewer than all of the legs 12. The hook 16 is configured for engaging the wall of the blood vessel into which the filter 10 is deployed and may be made of the same material as the leg 12 to which it is attached, or a different material, examples of which are provided above with respect to possible materials for the legs 12. The hook 16 may be formed with the leg 12 during manufacture, thus being integral therewith, or may be attached subsequent to formation of each by any attachment method known to one skilled in the art (e.g., welding, adhesive bonding, solvent bonding, etc.). In one embodiment, the hook contains a linear portion connected to an arcuate portion that terminates in a point, as shown and described in U.S. Pat. No. 6,258,026. In one embodiment, the arcuate member has a cross-sectional area smaller than the cross-sectional area of the leg 12, as shown and described in U.S. Pat. No. 6,258,026. Alternatively, the hooks can be those shown and described in U.S. Patent Publication Nos. 2005/0101982 and 2005/0131451, which are incorporated by reference in their entireties into this application.

A filament 14 connects the legs 12 along their length distal to the proximal end thereof attached to the hub 18, the filament 14 as shown in FIG. 1 traveling along a helical path down the longitudinal axis L of filter 10. The filament 14 may connect the legs 12 by being wrapped around the perimeter of the filter 10 such that the filament contacts each of the legs 12, but is not necessarily attached to each leg 12. Alternatively, the filament 14 may be attached to one or more legs 12 of the filter 10. Examples of ways in which filament 14 is attached to legs 12 in certain embodiments, depending on the materials utilized for the filament 14 and legs 12, include wrapping the filament one or more times around the leg 12, tying the filament 14 to the leg 12, heating the filament 14 adjacent to the leg 12 to create a bond therebetween, applying an adhesive to the filament 14 and/or the leg 12, applying a solvent to the filament 14 and/or the leg 12, etc. Of course, other possibilities for attaching the filament 14 to legs 12 known to one skilled in the art are also within the scope of this invention.

Variations on attachment of the filament 14 to the legs 12 include attaching the filament to each leg 12, to alternating legs 12, to every third leg 12, etc. Although a single filament 14 is shown traveling along a helical path down the longitudinal axis L of filter 10, other embodiments are also possible. For example, two or more filaments 14 could be spaced apart and arranged in a helical path down the longitudinal axis of filter 10, two or more filaments 14 could be arranged in opposing helical paths, etc. In addition, while the filament 14 is shown in FIG. 1 to originate at the proximal end of the filter 10 and terminate at a distal end of the filter 10, in another embodiment, the filament 10 could continue from the distal end of the filter 10 back toward the proximal end of the filter 10 in a similar or opposing helical path. The filament 14 may be made of suture material or any other material mentioned above as examples for possible materials for the legs 12.

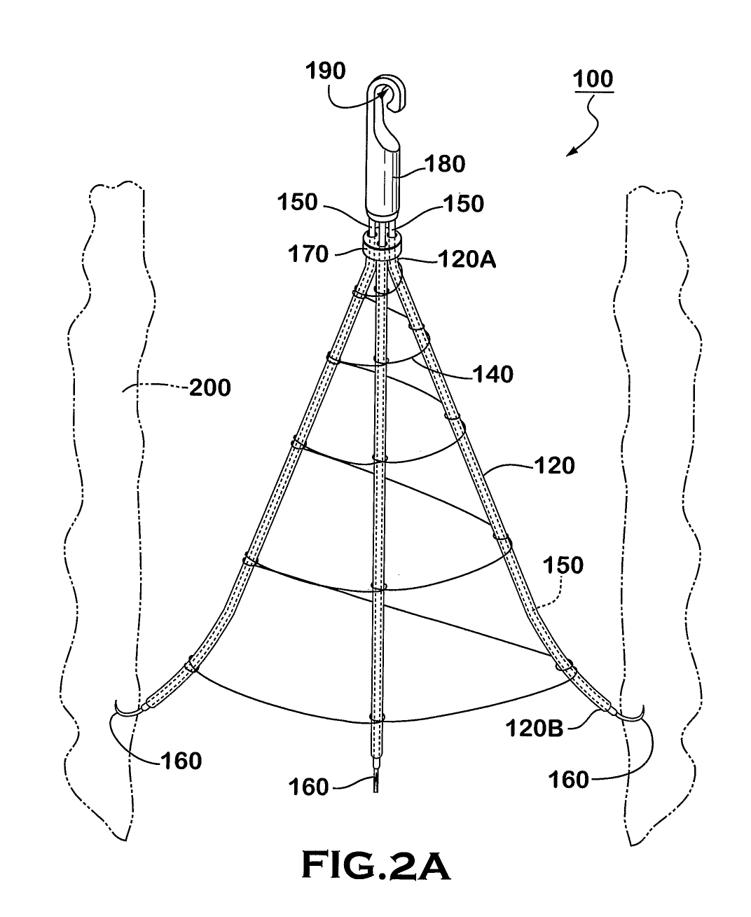

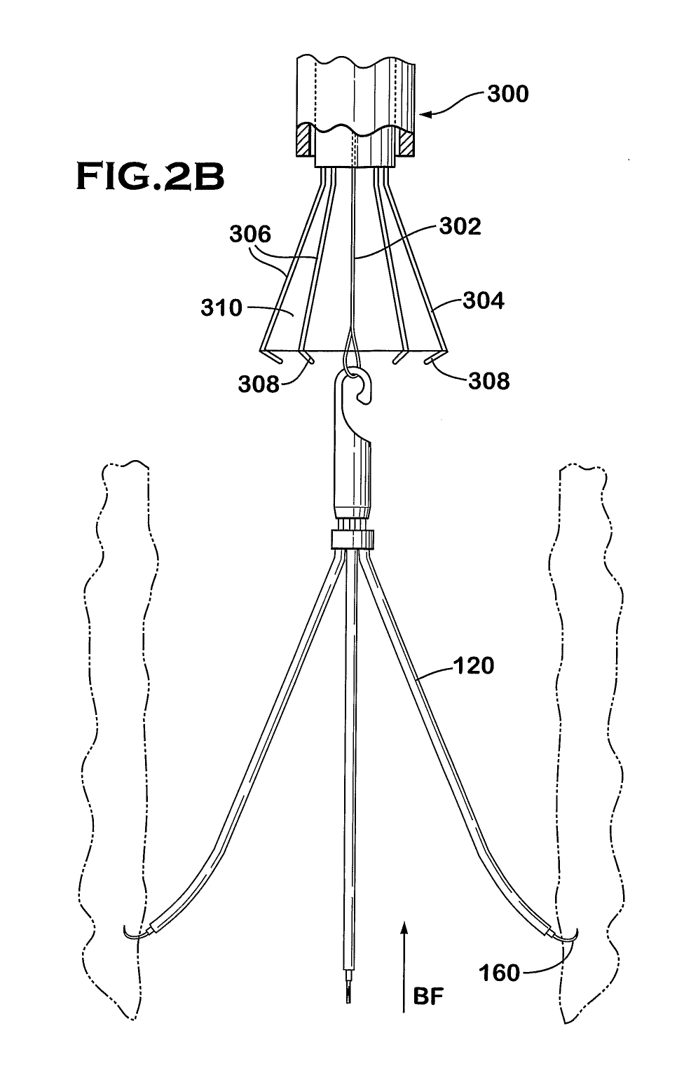

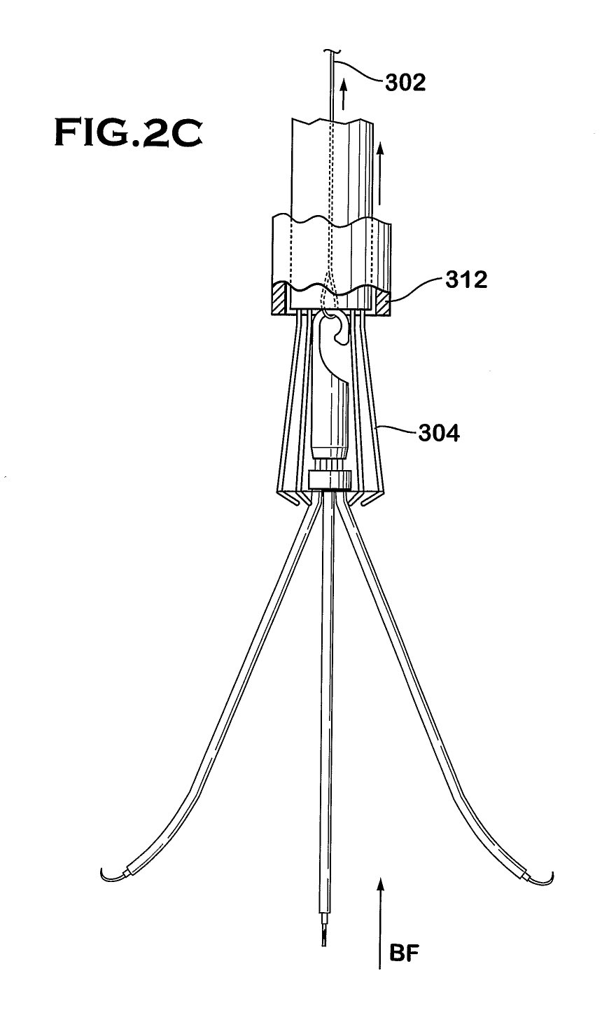

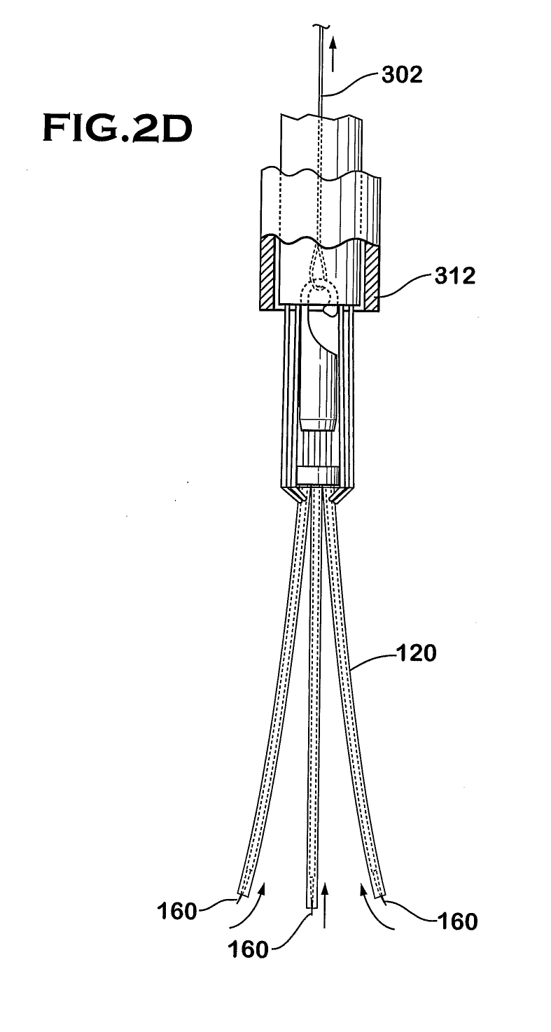

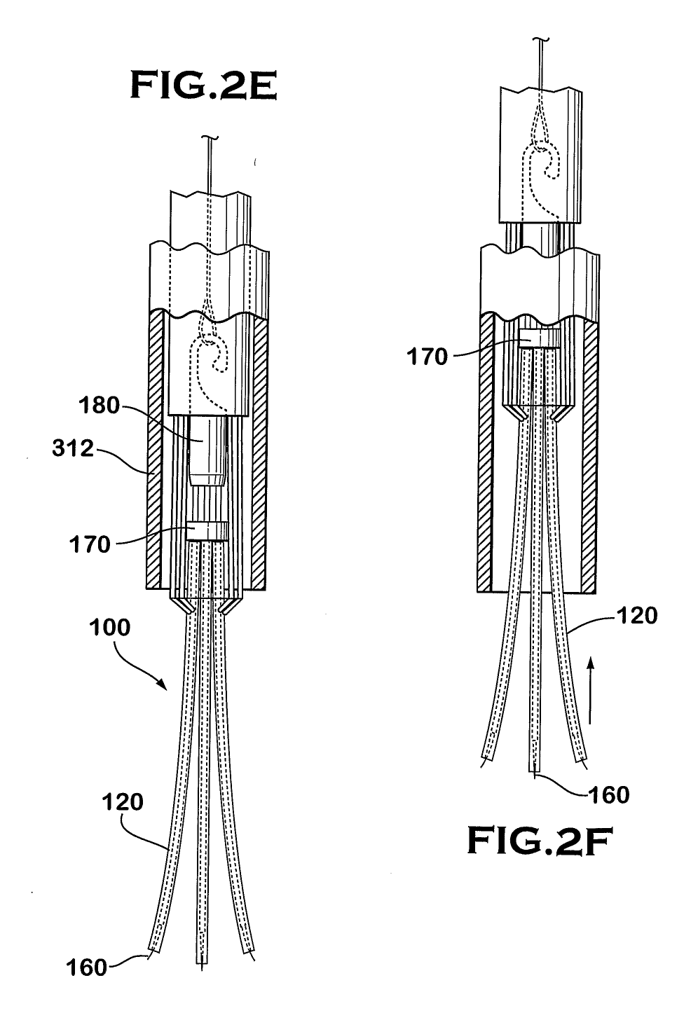

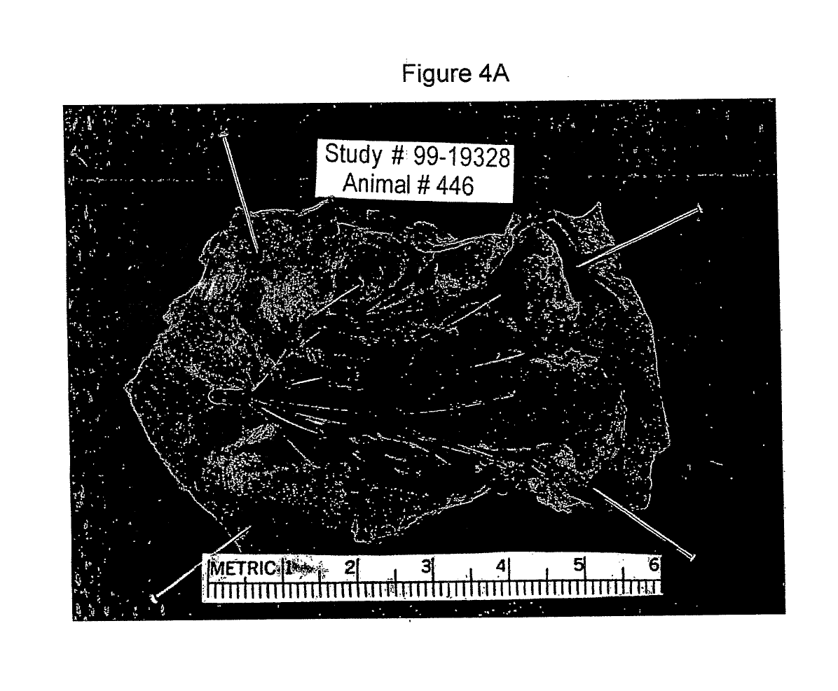

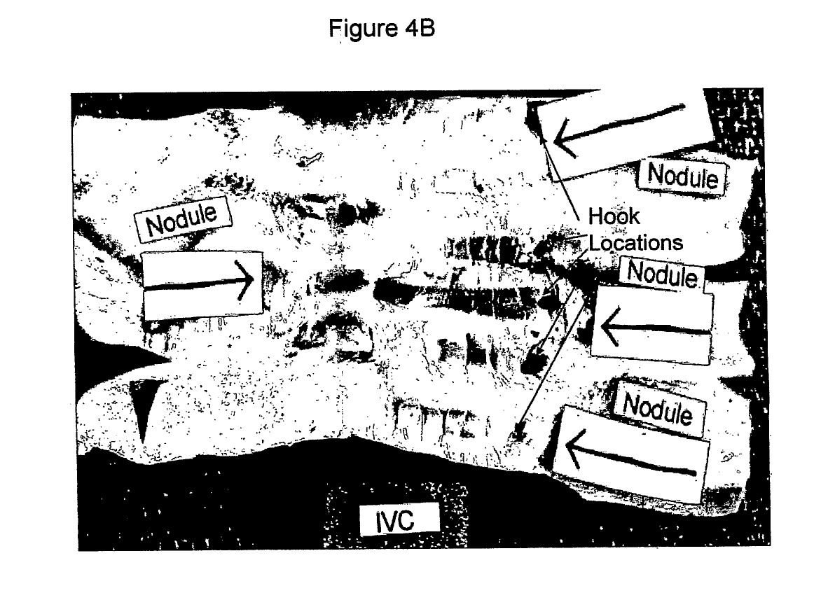

In an alternative embodiment, shown here in FIG. 2A, a filter 100 is provided that facilitates ease of retrieval. Filter 100 is provided with a plurality of tubes 120 that are hollowed from a proximal end 120A to a distal end 120B and which are fixed to boss portion 170. Disposed in each hollowed tube 120 is elongated member 150 provided with a hook 160 at its distal end. The elongated members 150 can be coupled to a hub portion 180, which can be provided with a snareable hook 190. The elongated members 150 are fixed to the hub 180, but are free to translate in the hollow tubes 120. The hooks 160 are of particular interest in that each hook is of sufficient size to maintain its arcuate configuration when an axial force along the longitudinal axis L-L away from each hook 160 is less than about 100 grams of force for each hook 160. However, as the axial force becomes greater than about 100 grams, each hook changes its arcuate configuration along a portion of a first radius of curvature to a portion of a second radius of curvature greater than the first radius. When the axial force is much greater than 100 grams, the hook 160 is pulled toward somewhat of a linear configuration, allowing each hook 160 to withdraw from the blood vessel wall 200 without substantial trauma to the blood vessel wall, which has been demonstrated for a similar hook (shown and described in U.S. Pat. No. 6,258,026) in at least one clinical study (FIGS. 4A and 4B).

As shown in FIG. 4A, a blood filter similar to a filter shown and described in U.S. Pat. No. 6,258,026 is explanted from an animal in order to study the traumatic effect of such filter on the blood vessel wall. As shown in FIG. 4B, the filter is removed to show the locations of the filter hooks after the hooks have been removed twelve weeks after implantation. Each location where the hooks have embedded into the vessel wall show what is believed to be insignificant trauma on the vessel wall. In the preferred embodiments, each of the hooks has a largest diameter on its arcuate portion of less than about 0.013 inches, preferably about 0.0085 inches and most preferably 0.0105 inches. Details of the hooks are shown and described in U.S. patent application Ser. No. 11/429,975, filed May 9, 2006, which application is incorporated by reference in its entirety into this application. Alternatively, as mentioned above, the hooks can be those shown and described in U.S. Patent Application Publication Nos. 2005/0101982 and 2005/0131451.

In the preferred embodiments described herein, it is believed that further reduction in trauma on the blood vessel wall during removal can be achieved by having the hooks 160 attached respectively to elongated members 150 so that they can be retracted at least partly into the hollowed tubes 120 prior to retracting the entire filter 100 proximally during a recovery or repositioning procedure of the filter 100.

Referring to FIGS. 2B-2F, a recovery (or repositioning) procedure is illustrated. As shown in FIG. 2B, the filter 100 is shown in a blood vessel over a certain amount of time in which a bio-resorbable filament has been resorbed, thereby leaving only the non-resorbable portion of the filter 200. A recovery cone device 300 is provided in which a snare member 302 extends beyond and out of a perimeter of a cone 304. The cone 304 is provided with claws 306, hooks 308, and a suitable connecting member 310 in the form of a polymeric mesh or cover such as, for example, polyurethane, Nylon, ePTFE or Kevlar. Once the snare member 302 has captured the snareable member 190, the snare 302 is moved downstream of the blood flow BF to pull the snareable member 190 into the cone 304. At this point, both the snare 302 and cone 304 are pulled downstream for a predetermined distance. The cone 304 is forced towards a smaller conic configuration (FIG. 2C) as it is being pulled inside a recovery catheter 312. Once the cone 304 reaches a certain distance, it remains stationary while the snare 302 is continued to be pulled downstream (FIG. 2D).

Because the cone 304 is generally stationary, it tends to force the boss portion 170 to remain stationary and therefore the hollow tubes 120 are also stationary, while the elongated members 150 are being pulled downstream by virtue of the connection between the members 150 to the snareable member 180. This has the effect of retracting the elongated members 150 and hooks 160 (which are now deformed towards a generally linear configuration) into the tubes 120. Continued movement of the snare 302 downstream at a predetermined point will also pull the cone 304 downstream into the catheter tube 312, shown here in FIGS. 2E and 2F, where the filter 100 is substantially pulled into the catheter tube 312. Alternatively, the recovery device may have a stop member formed between the snare 302 and cone 310 to limit the extent in which the snare 302 can be moved proximally relative to the cone as the filter is retracted into the catheter. The catheter tube 312 can be withdrawn proximally towards the operator to remove the catheter tube 312 and filter 100 from the blood vessel. The retractable members 150 may have a stop member (not shown) formed proximate the respective hooks 160 to prevent the snareable member 190 from pulling the retractable members 150 completely through the boss portion 170.

It is believed that the designs of the embodiments exemplarily described and shown in FIGS. 1 and 2A-2F are advantageous for many reasons. First, the filament 14 and the plurality of extending appendages provide for two levels of blood filtration similar to that of the two-conical filter baskets of U.S. Pat. No. 6,007,558, which is incorporated by reference in its entirety into this application. Second, the use of the filament 14 allows for a smaller profile when the filter is compacted into a delivery catheter. Third, the use of filament 14 that is bio-resorbable within a period of time, e.g., 60-180 days, allows for retrieval of the filter 10 with just its appendages without the complication of entanglement with the suture filaments. Fourth, the use of deformable hooks, whether or not retractable into hollow tubes, allows for little or no trauma to the wall of the blood vessel.

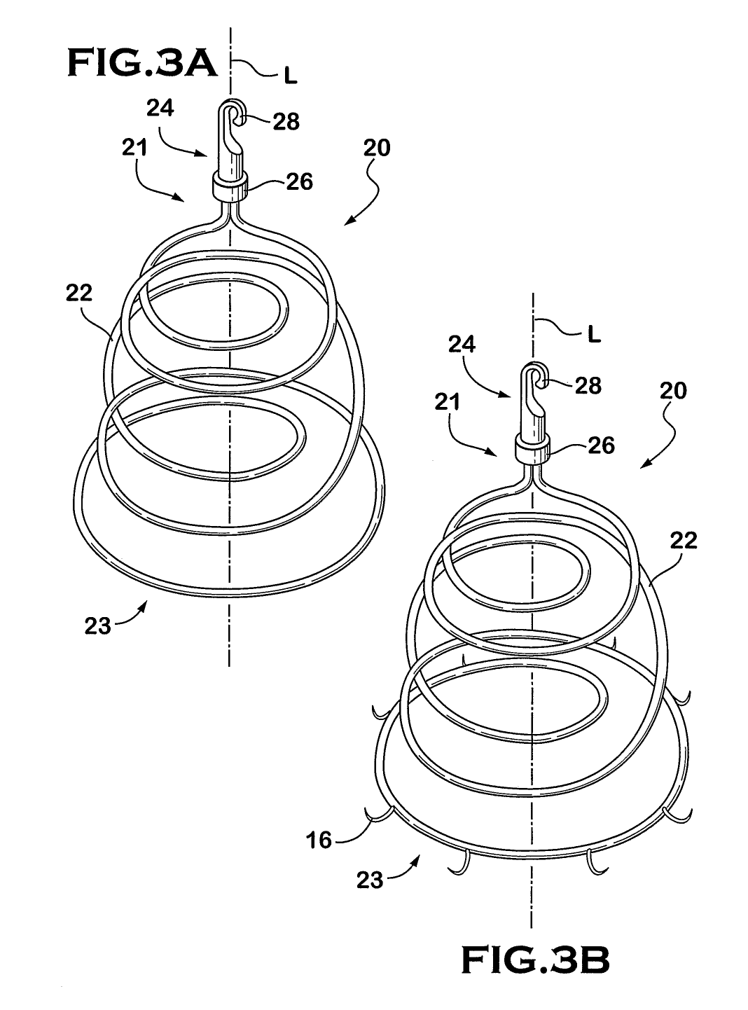

Referring now to FIGS. 3A and 3B, another embodiment of a filter is illustrated. Filter 20 includes an elongated wire member 22 that spans from a proximal end 21 of the filter 20 to a distal end 23 of the filter 20. The elongated wire member 22 has a proximal free end and a distal free end that are each positioned at the proximal end 21 of the filter 20 and are attached to a hub 24. In one embodiment, the proximal free end and distal free end of the elongated wire member 22 are joined together prior to attaching to the hub 24. The elongated wire member 22 is arranged helically along a longitudinal axis L such that successive windings of the wire member 22 along a first helical path expand radially from the proximal end 21 to the distal end 23 whereupon the elongated wire member travels back to the proximal end 21 from the distal end 23 in a second helical path in which successive windings of the wire member 22 radially decrease. Thus, the first helical path defines a first outer boundary that is conical in shape, expanding from the proximal end 21 to the distal end 23, and the second helical path defines a second outer boundary also having a conical shape, the second outer boundary being within the first outer boundary.

In another embodiment, the outer boundary of the first helical path is within the outer boundary of the second helical path. In other embodiments, the first and second helical paths define alternating outer perimeters (e.g., a winding or loop of the first helical path is smaller than the adjacent windings or loops of the second helical path, but the next winding or loop of the first helical path (in a distal direction) is greater than the adjacent windings or loops of the second helical path). In all embodiments, the filter 20 has an expanded configuration with an expanded perimeter following deployment in a blood vessel, such as shown in FIGS. 3A and 3B. For delivery of the filter 20 to a blood vessel, the filter 20 is compressed to a collapsed configuration, defining a collapsed perimeter smaller than the expanded perimeter.

The hub 24 includes a base portion 26 and a retrieval member 28. In the embodiment shown, the retrieval member 28 has a hook-like configuration, although as known to one skilled in the art, there are many possible forms for the retrieval member, such as, for example, a loop, rod, shaft, etc., depending on the form of the removal device to be utilized. One example of a retrieval member is disclosed in U.S. Pat. No. 6,156,055, which is incorporated by reference in its entirety into this application. The hub 24 and elongated wire member 22 may be made of a material, such as, for example, stainless steel, shape memory metals, shape memory alloys, super elastic shape memory metal alloys, metal alloys, linear elastic shape memory alloy, shape memory polymers, polymers, and combinations thereof. The hub 24 and/or elongated wire member 22 may also be made of a bio-resorbable material such as, for example, such as, for example, the materials shown and described in U.S. Pat. No. 6,287,332 and U.S. Patent Application Publication No. 2002/0004060, which are incorporated by reference in their entireties into this application.

In one embodiment, one or more hooks for anchoring the filter 20 to a blood vessel wall may be attached to the elongated wire member 22 along its length at any point among the first or second helical path. The hook or hooks may be attached by any attachment method known to one skilled in the art (e.g., welding, adhesive bonding, solvent bonding, etc.). In one embodiment, the hook contains a linear portion connected to an arcuate portion that terminates in a point, as shown and described in U.S. Pat. No. 6,258,026. In one embodiment, the arcuate member has a cross-sectional area smaller than the cross-sectional area of the wire member. In the embodiment shown in FIG. 3A, hooks 25 are attached to the elongated wire member 22 at the distal end 23.

This invention has been described and specific examples of the invention have been portrayed. While the invention has been described in terms of particular variations and illustrative figures, those of ordinary skill in the art will recognize that the invention is not limited to the variations or figures described. For example, other materials can be incorporated with the filter such as, for example, bio-active agents such as blood de-clotting agent (e.g., heparin, warfarin, etc.). The bio-active agents can be incorporated into the filaments. Other bio-active agents may include, but are not limited to substances such as, for example, anti-proliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; anti-proliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); anti-proliferative/antimitotic antimetabolites such as folic acid analogs (methotrexate), pyrimidine analogs (fluorouracil, floxuridine, and cytarabine), purine analogs and related inhibitors (mercaptopurine, thioguanine, pentostatin and 2-chlorodeoxyadenosine {cladribine}); platinum coordination complexes (cisplatin, carboplatin), procarbazine, hydroxyurea, mitotane, aminoglutethimide; hormones (i.e. estrogen); anti-coagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); anti-inflammatory: such as adrenocortical steroids (cortisol, cortisone, fludrocortisone, prednisone, prednisolone, 6α-methylprednisolone, triamcinolone, betamethasone, and dexamethasone), non-steroidal agents (salicylic acid derivatives i.e. aspirin; para-aminophenol derivatives i.e. acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives: (cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor receptor signal transduction kinase inhibitors; retenoids; cyclin/CDK inhibitors; HMG co-enzyme reductase inhibitors (statins); and protease inhibitors.

In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

What is claimed is:

1. A blood vessel filter, comprising:
a plurality of legs extending radially outward from a proximal portion of the filter along a longitudinal axis, the legs including a hook on a distal end thereof; and
a filament connecting the legs, the filament traveling in a first helical path around the legs along the longitudinal axis.

2. The blood vessel filter according to claim 1, wherein at least one of the legs and the filament comprises a nitinol wire.

3. The blood vessel filter according to claim 1, wherein at least one of the legs and filament comprises a suture material.

4. The blood vessel filter according to claim 1, wherein a retrieval member is attached to the proximal portion of the filter.

5. The blood vessel filter according to claim 1, wherein the filament is looped around successive legs along the helical path.

6. The blood vessel filter according to claim 1, wherein a portion of each of the legs comprises a bio-resorbable material.

7. The blood vessel filter according to claim 1, wherein the legs comprise a material selected from the group consisting of stainless steel, shape memory metals, shape memory alloys, super elastic shape memory metal alloys, metal alloys, linear elastic shape memory alloy, shape memory polymers, polymers, and combinations thereof.

8. The blood vessel filter according to claim 1, further comprising a second filament connecting the legs.

9. The blood vessel filter according to claim 8, wherein the second filament travels in a second helical path different from the first helical path.

10. The blood vessel filter according to claim 8, wherein the second filament travels in the first helical path and is spaced apart from the filament.

11. The blood vessel filter according to claim 1, wherein the hook comprises a linear portion connected to an arcuate portion that terminates to a point, the arcuate member having a cross-sectional area smaller than the cross-sectional area of a leg.

12. The blood vessel filter according to claim 1, wherein the plurality of legs comprises a first set of legs having a first length and a second set of legs having a second length different from the first length.

13. A blood vessel filter, comprising:
a plurality of hollow generally tubular members extending radially outward from a proximal portion of the filter along a longitudinal axis;
a plurality of elongated members that extend through respective hollow generally tubular members, the elongated member having a hook coupled to an end disposed radially away from the longitudinal axis; and
a filament connecting the legs and disposed about the longitudinal axis.

14. The blood vessel filter of claim 13, wherein at least one of the hooks comprises a cross-sectional area smaller than the smallest cross-sectional area of the elongated member.

15. The blood vessel filter of claim 13, wherein at least one of the hooks comprises a cross-sectional area generally equal to the smallest cross-sectional area of the elongated member.

16. The blood vessel filter of claim 13, wherein the elongated members are connected to a hub so that movement of the hub along the longitudinal axis relative to the hollow tubular members results in translation of the elongated members with respect to the tubular members.

17. A method of retrieving a blood filter having a plurality of hollow generally tubular members having distal ends extending radially outward from a proximal portion of the filter along a longitudinal axis with a plurality of elongated members that extend through respective hollow generally tubular members, the elongated member having a proximal portion and a distal portion having a hook coupled to an end disposed radially away from the longitudinal axis, the method comprising:
coupling a snare to a proximal portion of the elongated member;
contacting the tubular member with a catheter portion;
pulling at least a portion of each hook into each tubular member;
moving the distal ends of the tubular members toward the longitudinal axis; and
retracting the tubular members into the catheter portion.

18. A blood vessel filter, comprising an elongated wire member arranged helically along a longitudinal axis, wherein both a proximal free end of the elongated wire member and a distal free end of the elongated wire member are positioned at a proximal end of the filter, the elongated wire member radially expanding along a first helical path from the proximal free end of the elongated wire member to a distal end of the filter and radially decreasing along a second helical path from the distal end of the filter to the distal free end of the elongated wire member.

19. The blood vessel filter according to claim 18, wherein the proximal free end of the elongated wire member and the distal free end of the elongated wire member are joined together.

20. The blood vessel filter according to claim 18, further comprising a retrieval member attached to the proximal end of the filter.

21. The blood vessel filter according to claim 18, wherein the elongated wire member comprises nitinol.

22. The blood vessel filter according to claim 18, wherein the elongated wire member comprises a bio-resorbable material.

23. The blood vessel filter according to claim 18, wherein an outer boundary of the second helical path is within an outer boundary of the first helical path.

24. The blood vessel filter according to claim 18, further comprising at least one hook coupled to the elongated wire member.

25. The blood vessel according to claim 24, wherein the at least one hook comprises a linear portion connected to an arcuate portion that terminates to a point, the arcuate member having a cross-sectional area smaller than the cross-sectional area of the wire member.
